(19) United States
(12) Patent Application Publication
(10) Pub. No.: US 2010/0240957 A1
(43) Pub. Date: Sep. 23, 2010

(54) ACCESS PORT INCLUDING CENTERING FEATURE

(76) Inventor: Michael E. Abrams, New York, NY (US)

Correspondence Address: Tyco Healthcare Group LP;d/b/a Covidien, 555 Long Wharf Drive, Mail Stop 8-N1, Legal Department, New Haven, CT, 06511, US

(21) Appl. No.: 12/717,268

(22) Filed: Mar. 4, 2010

Related U.S. Application Data
(60) Provisional application No. 61/160,733, filed on Mar. 17, 2009.

(51) Int. Cl. A61B 1/32

(52) U.S. Cl. 600/201

(57) ABSTRACT

An access port includes a housing defining a longitudinal axis and having proximal and distal ends, and an interior wall defining a longitudinal opening adapted for passage of a surgical object, an object seal disposed in mechanical cooperation with the housing and being configured to create a substantially fluid-tight seal around a surgical object inserted through the object seal and a centering mechanism mounted to the housing. The centering mechanism includes at least one centering element extending at least in a general longitudinal direction within the longitudinal opening and a substantially annular ring mounted to the at least one centering element. The at least one centering element is positioned and dimensioned to engage the surgical object during passage of the object through the longitudinal opening and is capable of radial outward deflective movement relative to the longitudinal axis in response to an outward force exerted by the surgical object during eccentric manipulation of the surgical object. The annular ring is adapted for radial movement during corresponding radial movement of the at least one centering element upon eccentric manipulation of the surgical object, to thereby engage the interior wall and apply a generally inward force counteracting the outward force exerted by the surgical object tending to bias the surgical object toward a generally aligned position with respect to the longitudinal axis.

### CROSS REFERENCE TO RELATED APPLICATION

The present application claims the benefit of and priority to U.S. Provisional Application Ser. No. 61/160,733 filed on Mar. 17, 2009, the entire contents of which are incorporated herein by reference.

### BACKGROUND

1. Technical Field

The present disclosure relates to an access port which is adapted to allow the introduction of surgical instrumentation into a patient's body.

2. Description of the Related Art

In laparoscopic procedures, surgery is performed in the interior of the abdomen through a small incision; in endoscopic procedures, surgery is performed in any hollow viscus of the body through a narrow tube or cannula inserted through a small entrance incision in the skin. Laparoscopic and endoscopic procedures generally require that any instrumentation inserted into the body be sealed, i.e. provisions must be made to ensure that gases do not enter or exit the body through the incision as, for example, in surgical procedures in which the surgical region is insufflated. Moreover, laparoscopic and endoscopic procedures often require the surgeon to act on organs, tissue, and vessels far removed from the incision, thereby requiring that any instruments used in such procedures be relatively long and narrow.

For such procedures, the introduction of a tube into certain anatomical cavities such as the abdominal cavity is usually accomplished by use of a trocar assembly made up of a cannula assembly and an obturator assembly. Since the cannula assembly provides a direct passage for surgical instrumentation from outside the patient's body to access internal organs and tissue, it is important that the cannula assembly maintain a relatively gas-tight interface between the abdominal cavity and the outside atmosphere. The cannula assembly generally includes a cannula attached to a cannula housing containing a seal assembly adapted to maintain a seal across the opening of the cannula housing.

Since surgical procedures in the abdominal cavity of the body require insufflating gases to raise the cavity wall away from vital organs, the procedure is usually initiated by use of a Verres needle through which a gas such as CO₂ is introduced into the body cavity, thereby creating a pneumoperitoneum. The gas provides a positive pressure which raises the inner body wall away from internal organs, thereby providing the surgeon with a region within which to operate and avoiding unnecessary contact with the organs by the instruments inserted through the cannula assembly. An obturator of the obturator assembly is inserted into the cannula assembly and used to puncture the abdominal wall. Following removal of the obturator assembly from the cannula assembly, laparoscopic or endoscopic surgical instruments may be inserted through the cannula assembly to perform surgery within the abdominal cavity.

Generally in the context of insufflatory surgical procedures, there are two sealing requirements for cannula assemblies. The first requirement is to provide a substantially fluid-tight seal when an instrument is not being introduced into or is not already present in the cannula. The second requirement is to provide a substantially fluid-tight seal when an instrument is being introduced into or is already present in the cannula. Additionally, as endoscopic and laparoscopic surgical procedures and techniques have advanced, it has become desirable to accommodate surgical instrumentation of varying outside diameters through a single cannula assembly in a given surgical procedure, thereby minimizing the number of cannula required and facilitating efficiency in the surgical procedure.

### SUMMARY

In accordance with a preferred embodiment, an access port includes a housing defining a longitudinal axis and having proximal and distal ends, and an interior wall defining a longitudinal opening adapted for passage of a surgical object, an object seal disposed in mechanical cooperation with the housing and being configured to create a substantially fluid-tight seal around a surgical object inserted through the object seal and a centering mechanism mounted to the housing. The centering mechanism includes at least one centering element extending at least in a general longitudinal direction within the longitudinal opening and a substantially annular ring mounted to the at least one centering element. The at least one centering element is positioned and dimensioned to engage the surgical object during passage of the object through the longitudinal opening and is capable of radial outward deflective movement relative to the longitudinal axis in response to an outward force exerted by the surgical object during eccentric manipulation of the surgical object. The annular ring is adapted for radial movement during corresponding radial movement of the at least one centering element upon eccentric manipulation of the surgical object, to thereby engage the interior wall and apply a generally inward force counteracting the outward force exerted by the surgical object tending to bias the surgical object toward a generally aligned position with respect to the longitudinal axis.

In one embodiment, a plurality of centering elements is provided. An annular ring may be mounted to move within the longitudinal opening of the housing. The centering elements each may include a proximal end segment secured to the housing and a distal end segment secured to the annular ring. The annular ring may be adapted for radial movement and longitudinal movement with respect to the longitudinal axis. The centering elements may be coaxially arranged about the longitudinal axis. The centering elements may be each dimensioned to have an intermediate bow segment between the proximal and distal end segments. The bow segment may define a substantially curved configuration. The object seal may define a substantially conical segment with the object seal being at least partially disposed within the centering elements.

In another embodiment, a surgical cannula assembly includes a cannula housing, a cannula member extending from the cannula housing, and defining a longitudinal axis and having a longitudinal opening for reception and passage of a surgical object, a plurality of centering elements mounted to the cannula housing in coaxial arrangement with the longitudinal axis and an object seal at least partially disposed within an inner boundary defined within the centering elements and being adapted to create a substantially fluid-tight seal around the surgical object. The centering elements may be positioned and dimensioned to engage the surgical object during passage of the object through the longitudinal opening and capable of radial outward deflective movement relative to the longitudinal axis from an initial position to a radial outward position in response to an outward force exerted by the surgical object during eccentric manipulation of the surgical object. The centering elements may be normally biased toward the initial position to bias the surgical object toward a generally aligned position with respect to the longitudinal axis.

The object seal may define a generally tapered segment, e.g., a generally conical segment extending along the longitudinal axis. A substantially annular ring may be mounted to the centering elements. The annular ring may be adapted for radial movement during corresponding radial movement of the centering elements upon eccentric manipulation of the surgical object. The ring may be adapted to move in a radial and longitudinal direction relative to the longitudinal axis. The ring may be adapted to engage an interior wall of the cannula housing and apply a generally inward force counteracting an outward force exerted by the surgical object during eccentric movement of the surgical object, to thereby tend to bias the surgical object toward a generally aligned position with respect to the longitudinal axis.

### BRIEF DESCRIPTION OF THE DRAWINGS

Various embodiments of the present disclosure are described herein with reference to the drawings wherein:

FIG. 1 is a longitudinal cross-sectional view of the access port illustrating initial insertion of an instrument.

FIG. 2 is a view similar to the view of FIG. 1 illustrating eccentric manipulation of the instrument within the access port and relative to the longitudinal axis of the access port;

FIG. 3 is a view similar to the view of FIG. 1 illustrating the instrument in substantial alignment with the longitudinal axis; and

FIG. 4 is a longitudinal cross-sectional view of the access port illustrating a relatively large instrument introduced within the access port.

### DETAILED DESCRIPTION

The access port of the present disclosure, either alone or in combination with a cannula assembly, provides a substantially fluid-tight seal between a body cavity of a patient and the outside atmosphere. The access port of the present disclosure is configured to receive instruments of varying diameter. The centering mechanism includes ribs which assist in maintaining a substantially symmetrical position of a surgical instrument with respect to a longitudinal axis during insertion into the access port and cannula assembly. The ribs also help maintain the substantially symmetrical position of an instrument disposed within the access port while the instrument is disposed therethrough.

The access port of the present disclosure contemplates the introduction of various types of instrumentation adapted for insertion through a trocar and/or cannula assembly while maintaining a substantially fluid-tight interface about the instrument to help preserve the atmospheric integrity of a surgical procedure from gas and/or fluid leakage. Specifically, the access port includes at least two ribs which bias an instrument entering the port such that the instrument enters the port or, is normally biased toward a substantially symmetrical relation with the longitudinal axis. This feature of the present disclosure minimizes the entry and exit of gases and/or fluids to/from the body cavity. Examples of instrumentation include, but are not limited to, clip appliers, graspers, dissectors, retractors, staplers, laser probes, photographic devices, endoscopes and laparoscopes, tubes, and the like. Such instruments will collectively be referred to as “instruments” or “instrumentation.”

In the following description, as is traditional, the term “proximal” refers to the portion of the device closer to the operator while the term “distal” refers to the portion of the device farther from the operator.

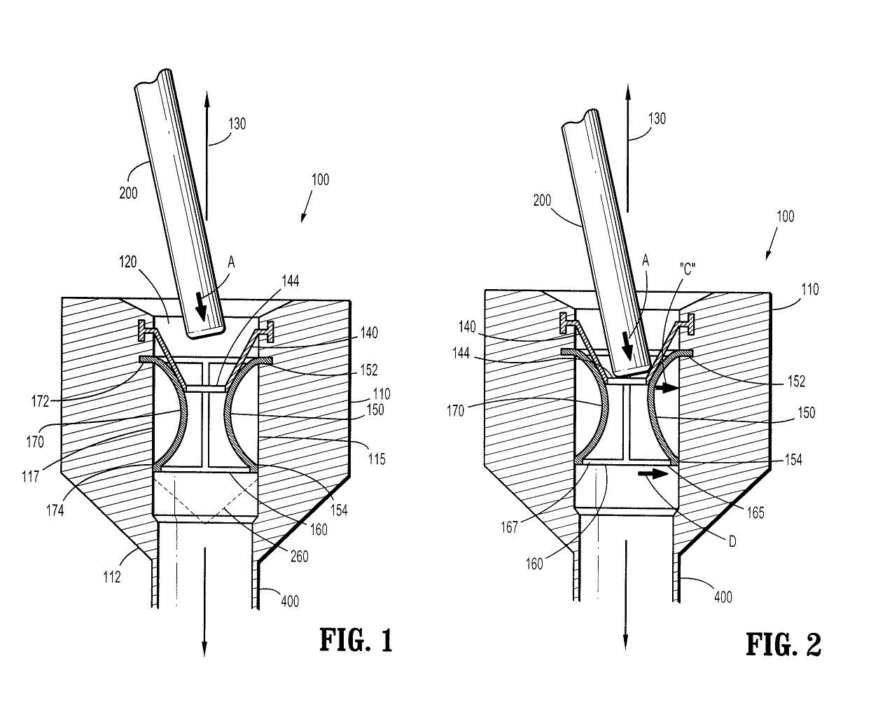

Referring now to the drawings, FIG. 1 shows access port 100 including a housing 110 defining a longitudinal axis 130. Housing 110 includes an orifice seal 140 having an aperture 144 therein. Orifice seal 140 may be made from a low durometer elastomer and/or include a hydrophilic coating. Orifice seal 140 may be a conical or tapered seal extending along the longitudinal axis 130. Opposing ribs 150 and 170 are rigidly attached to respective inner surfaces 115, 117 of housing 110 adjacent their respective proximal ends 152, 172 and attached to a ring 160 adjacent their respective distal ends 154, 174. Ring 160 is free of engagement with housing 110 and is thus free to move within the housing in both a radial and a longitudinal direction. Ribs 150, 170 and ring 160 form a centering mechanism tending to bias the surgical object into general alignment with the longitudinal axis 130. Ribs 150, 170 may be formed of any material having sufficient resiliency to permit deflection and return to its initial position. Ribs 150, 170 may be secured to an internal surface of housing 110 by conventional means including adhesive, cements, pins, fasteners or the like. Ring 160 may be secured to ribs 150, 170 in a similar manner. Suitable materials may include polymeric material, spring steel, titanium or the like. Ring 160 may be formed of a more rigid material. Ring 160 may define a diameter substantially approximating the internal dimension of inner wall 117.

A distal end 112 of housing 110 is shown monolithically formed with a cannula assembly 400. Access port 100 may also be configured to mechanically engage cannula assembly 400 in a variety of ways including, but not limited to, through a bayonet lock or threaded connection. Access port 100 and/or cannula assembly 400 may include a second seal 260 (shown in phantom) which provides a substantially fluid-tight seal in the absence of a surgical instrument passing therethrough.

While two ribs 150 and 170 are shown, it is envisioned and within the scope of the present disclosure that access port 100 includes more (e.g., four ribs at 90 degree radial intervals) or fewer than two ribs.

Opposing ribs 150, 170 may be bow-like in its normal state such that the gap distance between opposing ribs 150 and 170 at its narrowest point is slightly less than the smallest diameter instrument which is likely to be inserted into housing 110. For example, if the minimum diameter instrument which is likely to be introduced into housing 110 is about 5 mm, the gap distance between opposing ribs 150 and 170, at its narrowest point, could be about 4.5 mm in an at-rest position. Opposing ribs 150, 170 may also be configured such that in a flexed position, opposing ribs 150 and 170 can accommodate instruments having a diameter up to about 12 mm. Thus, access port 100 may be adapted to receive surgical instrumentation having a diameter in the range of about 5 mm to about 12 mm. The capability of access port being adapted to accommodate smaller and larger diameter instruments is also envisioned.

Opposing ribs 150, 170 and ring 160 cooperate to assist in maintaining a substantially symmetrical relation of a surgical instrument 200 with respect to a longitudinal axis 130, thus, minimizing of leakage of fluids between orifice seal 140 and the object, e.g., once disposed through housing 110, surgical instrument 200 is radially held in place, substantially symmetrical about longitudinal axis 130 via opposing ribs 150, 170 and ring 160. The operation of ribs 150, 170 and ring 160 before, during and after insertion of a surgical instrument into housing 110 will be described more fully below.

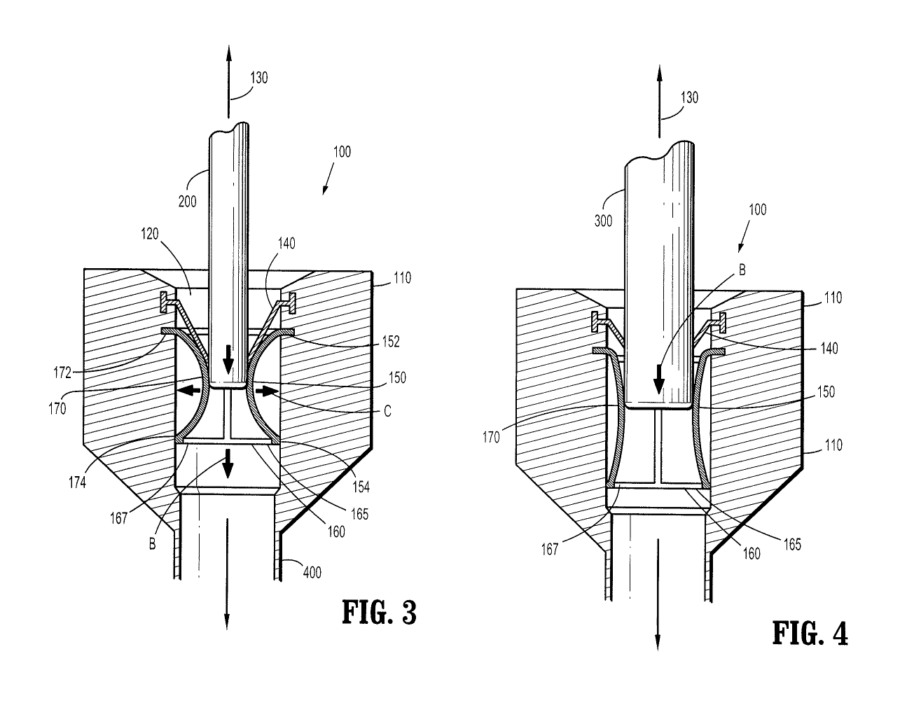

The use of access port 100 will now be described in detail with reference to FIGS. 1-4. FIG. 1 shows instrument 200 being introduced into a channel 120 defined within housing 110 of access port 100 in an asymmetrical direction about longitudinal axis 130 (arrow A). Asymmetrical may be interpreted as at least including offset, angulated, lateral or the like with respect to the longitudinal axis 130. Before insertion of instrument 200, ribs 150, 170 and ring 160 are in an initial at-rest position, as shown in FIG. 1. As instrument 200 is further advanced distally in the direction of arrow A, instrument 200 contacts rib 150. Upon contacting rib 150, as shown in FIG. 2, instrument 200 exerts both radial and longitudinal forces force on rib 150. Since rib 150 is rigidly attached to inner surface 115 of housing 110 adjacent proximal end 152, the force applied to rib 150 causes rib 150 to bow or deflect in the direction of arrow C which initially also causes translation of ring 160 in an axial direction, and imparts radial movement of the ring 160 relative to the longitudinal axis 130 and toward interior wall 115. Upon radial movement of ring 160 a predetermined distance “d”, ring 160 is forced into interior wall 115 in secured relation therewith through, e.g., a frictional relationship created between ring 160 and inner wall 115. With ring 160 secured relative to interior wall 115, the ring 160 may no longer translate in the axial direction. As a result, a counterforce is created within rib 150, biasing the rib 150 and the surgical object toward an aligned position with respect to the longitudinal axis. Once the surgical object is aligned, the surgical object may be advanced through aperture 144 of orifice seal 140 creating a fluid-tight relationship around instrument 200, as depicted in FIG. 3. If alignment is maintained, the surgical object may be advanced with ribs 150, 170 deflecting to cause corresponding axial movement of ring 160. For example, when an even load is applied to ribs 115, 117 coaxial arrangement of ring 160 with respect to longitudinal axial 130 thus permitting the ring 160 and instrument 200 to translate in an axial direction. However, if during any time, the surgical object is laterally manipulated or angulated relative to the longitudinal axis, ribs 150, 170 will deflect causing corresponding radial movement of ring 160 into engagement with inner wall 115. In this position, advancing movement of the surgical object is substantially prevented until the surgical object is in general alignment with the longitudinal axis 130 and, e.g., equal forces are applied to ribs 150, 170.

Ring 160 may include a textured outer surface, e.g., such as ribs, protrusions, teeth or the like to facilitate engagement with the interior wall 115 of inner wall 115. Ring 160 may also have an elastomeric outer surface to facilitate frictional engagement with interior wall 115. Interior wall 115 may include similar surfaces.

FIG. 4 illustrates an instrument 300 having a relatively large diameter (e.g., about 12 mm). Instrument 300 causes ribs 150 and 170 to bow radially outward, causing ring 160 to be displaced distally as instrument 300 is inserted into housing 110 in a substantially symmetrical manner with respect to longitudinal axis 130. Although the radial displacement of ribs 150 and 170 and the distal displacement of ring 160 would be greater than in the case of instrument 200, ribs 150 and 170 and ring 160 would operate in substantially the same manner. Instrument 300 would be held in place substantially symmetrical about longitudinal axis 130 and ribs 150, 170 and ring 160 would assist in minimizing asymmetrical insertion of instrument 300 and would also assist in minimizing movement of instrument 300 away from longitudinal axis 130.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An access port comprising:
a housing defining a longitudinal axis and having proximal and distal ends, the housing having an interior wall defining a longitudinal opening adapted for passage of a surgical object;
an object seal disposed in mechanical cooperation with the housing, the object seal configured to create a substantially fluid-tight seal around a surgical object inserted through the object seal; and
a centering mechanism mounted to the housing, the centering mechanism including:
at least one centering element extending at least in a general longitudinal direction within the longitudinal opening, the at least one centering element positioned and dimensioned to engage the surgical object during passage of the object through the longitudinal opening and capable of radial outward deflective movement relative to the longitudinal axis in response to an outward force exerted by the surgical object during eccentric manipulation of the surgical object; and
a substantially annular ring mounted to the at least one centering element, the annular ring adapted for radial movement during corresponding radial movement of the at least one centering element upon eccentric manipulation of the surgical object, to thereby engage the interior wall and apply a generally inward force counteracting the outward force exerted by the surgical object tending to bias the surgical object toward a generally aligned position with respect to the longitudinal axis.

2. The access port of claim 1 including a plurality of centering elements.

3. The access port of claim 2 wherein the annular ring is mounted to move within the longitudinal opening of the housing.

4. The access port of claim 3 wherein the centering elements each include a proximal end segment secured to the housing and a distal end segment secured to the annular ring.

5. The access port of claim 4 wherein the annular ring is adapted for radial movement and longitudinal movement with respect to the longitudinal axis.

6. The access port of claim 4 wherein the centering elements are coaxially arranged about the longitudinal axis.

7. The access port according to claim 4 wherein the centering elements are each dimensioned to have an intermediate bow segment between the proximal and distal end segments, the bow segment defining a substantially curved configuration.

8. The access port of claim 4 wherein the object seal defines a substantially conical segment, the object seal at least partially disposed within the centering elements.

9. A surgical cannula assembly, which comprises:
a cannula housing;
a cannula member extending from the cannula housing, the cannula housing and the cannula member defining a longitudinal axis and having a longitudinal opening for reception and passage of a surgical object;
a plurality of centering elements mounted to the cannula housing in coaxial arrangement with the longitudinal axis, the centering elements positioned and dimensioned to engage the surgical object during passage of the object through the longitudinal opening and capable of radial outward deflective movement relative to the longitudinal axis from an initial position to a radial outward position in response to an outward force exerted by the surgical object during eccentric manipulation of the surgical object, the centering elements normally biased toward the initial position to bias the surgical object toward a generally aligned position with respect to the longitudinal axis; and
an object seal at least partially disposed within an inner boundary defined within the centering elements, the object seal configured to create a substantially fluid-tight seal around the surgical object.

10. The surgical cannula assembly of claim 9 wherein the object seal defines a generally tapered segment extending along the longitudinal axis.

11. The surgical cannula assembly of claim 10 wherein the object seal defines a generally conical segment.

12. The surgical cannula assembly of claim 9 including a substantially annular ring mounted to the centering elements, the annular ring adapted for radial movement during corresponding radial movement of the centering elements upon eccentric manipulation of the surgical object.

13. The surgical cannula assembly according to claim 12 wherein the ring is adapted to move in a radial and longitudinal direction relative to the longitudinal axis.

14. The surgical cannula assembly according to claim 13 wherein the ring is adapted to engage an interior wall of the cannula housing and apply a generally inward force counteracting an outward force exerted by the surgical object during eccentric movement of the surgical object, to thereby tend to bias the surgical object toward a generally aligned position with respect to the longitudinal axis.
